(19) United States
(12) Patent Application Publication
(10) Pub. No.: US 2020/0155789 A1
(43) Pub. Date: May 21, 2020

(54) SYSTEM AND METHOD FOR DETERMINING THE EFFICACY OF MUSIC THERAPY

(71) Applicant: Dementia Society of America, Doylestown, PA (US)

(72) Inventor: Kevin William Jameson, Doylestown, PA (US)

(21) Appl. No.: 16/685,414

(22) Filed: Nov. 15, 2019

Related U.S. Application Data
(60) Provisional application No. 62/767,640, filed on Nov. 15, 2018.

(51) Int. Cl. A61M 21/00; G16H 10/60; G16H 20/70; G16H 50/30

(52) U.S. Cl. CPC A61M 21/00; G16H 10/60; A61M 2021/0027; G16H 50/30; G16H 20/70

(57) ABSTRACT

Methods, systems, and apparatus, including computer programs encoded on computer storage media, for determining an efficacy of music therapy are provided. An example system initiates a music therapy session for a patient on a music therapy device. The system provides the patient with a predefined type of music during the music therapy session at a pre-defined volume. The system ends the music therapy session. The system then receives a request to determine an efficacy of music therapy sessions for the patient for a defined time period and obtains personal and listening metrics associated with the patient's music therapy sessions for the defined time period. The system correlates the personal and listening metrics with the patient's medical metrics for the defined time period in order to determine the efficacy of music therapy sessions for the patient.

### CROSS-REFERENCE TO RELATED APPLICATION

This application claims priority to U.S. Application Ser. No. 62/767,640, filed on Nov. 15, 2018, which is incorporated by reference in its entirety.

### BACKGROUND

Music has been shown to reduce stress and anxiety in people, especially those living with dementia or other chronic diseases. For example, empirical evidence indicates that music can help “awaken” people with dementia who have been thought to be unreachable. If a dementia patient hears a tune that they know from their past, often they light up and sing along with the words.

Healthcare providers therefore provide music as therapy for patients. Music therapy is the use of music to address the physical, emotional, cognitive, and social needs of patients.

Patients who participate in music therapy programs are often observably more relaxed and in better moods after listening to music. These music therapy sessions tend to improve patients' overall well-being.

### SUMMARY

This specification describes technologies for determining the efficacy of music therapy. These technologies generally involve determining changes to a patient's overall health and healthcare metrics, including medication dosages, due to music therapy sessions.

An example system determines the efficacy of music therapy using listening and patient metrics provided by music therapy devices. The metrics from the music therapy devices can be correlated with the patients' healthcare metrics to determine the overall benefits of music therapy sessions to the patients. For example, after several music therapy sessions, a patient may require less medication than the patient normally requires when he or she does not have music therapy sessions.

In general, one innovative aspect of the subject matter described in this specification can be embodied in methods that include the actions comprising: initiating a music therapy session for a patient on a music therapy device, the music therapy device configured to execute a music therapy session that includes playing a predefined type of music for a predefined amount of time at a specified volume; providing the patient with the predefined type of music during the music therapy session; ending the music therapy session; storing personal and listening metrics for the music therapy session; receiving a request to determine an efficacy of music therapy sessions for the patient for a defined time period; obtaining personal and listening metrics associated with the patient's music therapy sessions for the defined time period; and determining the efficacy of music therapy sessions for the patient by correlating the personal and listening metrics with the patient's medical metrics for the defined time period is disclosed.

Other embodiments of this aspect include corresponding computer systems, apparatus, and computer programs recorded on one or more computer storage devices, each configured to perform the actions of the methods. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination.

Initiating a music therapy session for a patient on a music therapy device includes receiving an indication of an initial mood of the patient. Ending a music therapy session includes identifying a post-music therapy mood of the patient. The method further comprises: determining a change in a mood of a patient attributable to the music therapy session based on a difference between the initial mood of the patient and the post-therapy mood of the patient. Determining the efficacy of music therapy for the patient comprises correlating the patient's change in mood with medical metrics. Medical metrics comprise medications and dosages that the patient is currently taking, sleep patterns, eating habits, or daily activity habits. Personal metrics comprise, for each music therapy session in the defined period of time: heart rate data, mood data, or blood pressure data. Listening metrics comprise for each music therapy session in the defined period of time: the date and time of day of the music therapy session, the duration of the music therapy session, the genre of music to which the patient listened, the location of the music therapy session, who engaged with the patient to initiate and/or end the music therapy session, and the average volume at which the patient listened to the music. Providing the patient with the predefined type of music during the music therapy session comprises: accessing a preferred final music volume level of the patient; initializing the music therapy device with music at a low volume automatically; and incrementally increasing the volume on pre-defined intervals until the music volume matches the preferred final music volume level of the patient. The method further comprises: providing information regarding the efficacy of the music therapy sessions for display to a user.

A music therapy and healthcare correlation system may provide patients with music therapy sessions and determine the changes to patient metrics, including the patients mood, and overall medical requirements that can be attributable to the music therapy sessions. Although music has observable benefits to individual patients, scientific studies cannot yet conclusively link music therapy and true health benefits for patients. Thus, caregivers do not know exactly how (or, which) music affects patients' requirements for traditional medical treatments including psychotropic drug dosages.

Unlike conventional music therapy systems, the music therapy and healthcare correlation system of this specification determines patient metrics along with music listening metrics from a music therapy device used by a patient during music therapy sessions. The system then aggregates and correlates metrics received from the music therapy device with the patient's healthcare data and metrics in order to determine the overall health benefits of music therapy to the patient. Correlations and inferences made from these metrics and data can help inform future treatments and medical care for the patient.

The details of one or more implementations of the subject matter of this specification are set forth in the accompanying drawings and the description below. Other features and aspects of the subject matter will become apparent from the description, the drawings, and the claims.

### BRIEF DESCRIPTION OF THE DRAWINGS

FIG. 1 shows an example music therapy and healthcare correlation system.

FIG. 2 illustrates a startup user interface for a music therapy device to initiate a music therapy session.

FIG. 3 illustrates a user interface for adding a new music therapy patient profile to the music therapy application.

FIG. 4 illustrates a user interface for choosing an existing music therapy patient.

FIG. 5 illustrates a music therapy session initialization for a specific user.

FIG. 6 illustrates a user interface of the music therapy device during a music therapy session.

FIG. 7 illustrates a user interface of the music therapy device at the end of a music therapy session.

FIG. 8 is a flowchart of an example process for determining an efficacy of music therapy sessions for a patient.

Like reference numbers and designations in the various drawings indicate like elements.

### DETAILED DESCRIPTION

This specification generally describes music therapy and healthcare correlation systems and methods for determining the efficacy of music therapy.

Although music therapy is available in many nursing homes, assisted-living facilities, and hospitals, currently music therapy devices are hard to use or require many steps to initiate therapy sessions. Often the devices have small controls that are hard for older adults to see and manipulate. The devices also regularly use obsolete technology, need to have music loaded on them, and use non-intuitive processes. For these reasons, conventional music therapy programs are not successfully implemented in many places.

Even so, music therapy can provide enormous value to individuals if implemented correctly. In order to provide music therapy sessions to patients, caregivers need to have simple music-playing devices that are easy to operate and customizable for many different people. The music therapy and healthcare correlation system described in this specification provides music therapy devices that display user interfaces, which are intuitive, easy to use, and can accommodate multiple patient profiles.

The system collects data and metrics about patients and the music therapy sessions while the patients are using the devices. The system correlates and integrates the data and metrics with medical information of patients to effectively measure the actual medical benefits of music therapy to patients.

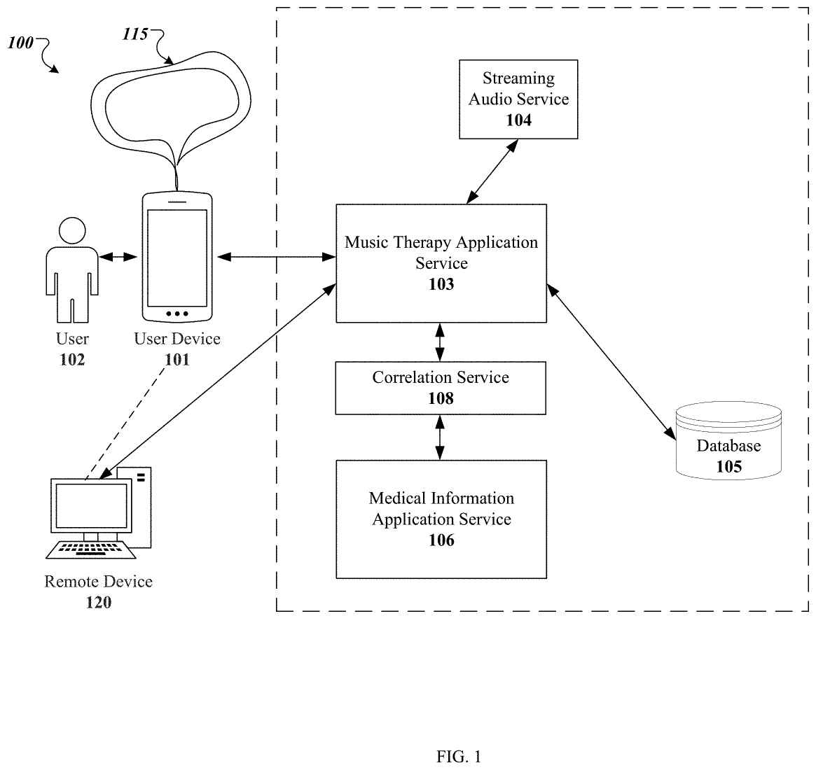

FIG. 1 shows an example music therapy and healthcare correlation system 100. The system 100 can be implemented as computer programs on one or more computers in one or more geographic locations, in which the systems, components, and techniques described below can be implemented.

The system includes a user device 101 configured to be used by a user 102 to execute a music therapy application from an application service 103. The music therapy application may be a mobile application, a web application, or a desktop application. The application may have specific functionality that executes on the client, e.g., the user device 101, without needing to access the music therapy application service 103. Other functionality may send requests from the client to a server of the music therapy application service. The music therapy application service will send responses to the request back to the client device.

Since most participants in music therapy are older adults with chronic diseases, such as dementia, music therapy devices need to be easy-to-use and are typically operated by a caregiver who has a lack of sophistication with computing devices. Often, for music therapy sessions, there are two users, an initial user, or caregiver who initiates music therapy sessions on behalf of patients and a listening user, or patient who participates in the music therapy session, but does not actively operate the music therapy device 101.

The user music therapy device 101 can be a smartphone, a portable computer, or another type of electronic device that executes applications. The music therapy device 101 may be attached to a breakaway lanyard 115 that can hang around a user's 102 neck so that the user does not lose the device.

Each music therapy device 101 is configured to execute a music therapy application that provides music therapy to patients.

The music therapy application is an application that plays a type of music for a particular patient for a predefined amount of time at a predefined volume. The music therapy application can be configured with multiple profiles for multiple different patients. Each profile can contain the default listening preferences of a particular patient including: music genres or channels preferred by the user or, deemed therapeutically most effective; the volume at which the patient prefers to listen to music, and the preferred length of time of music therapy sessions for the patient. The music therapy application is configured to add and update patient profiles as well as initiate, provide, and end music therapy sessions.

In order to provide music therapy sessions, the music therapy application provides music to a user device of a user. In some implementations, the music therapy application can provide music from a streaming music service 104 and provides predefined channels of different genres for a user to choose as a listening preference. For example, the music therapy application obtains data for music from a server system of the streaming music service 104 and play the music in real time. In some implementations, the music therapy application can provide music that is stored in a user device or an external memory coupled to the user device. For example, the music therapy application can access data for music at an internal memory of a user device or an external memory coupled to the user device, and play the music upon a request of a user. In some implementations, the music therapy application can provide music that is stored in a cloud computing platform. For example, the music therapy application allow a user device to access a cloud computing platform that stores data for music and download the data before playing the music. Thus, the music therapy application is not limited to be used for providing a music therapy session from a streaming service, but can be used for providing a music therapy session from any suitable types of stored media.

In some implementations, the music therapy application uses a streaming audio service 104 such as Apple Music, Spotify, Pandora, or Google Play Music. Music stations can be configured for users, e.g., patients, and for different music genres. A user may specify a particular music channel or genre. In some implementations, the user may specify a particular music channel or genre that corresponds to an existing channel or genre of the streaming music service or aggregates channels or genres from the streaming music service. That is, the new music channel or genre can be a pre-defined music channel or genre. The pre-defined music channel or genre can be provided by the streaming audio service 104 or stored in a user device. In some implementations, the particular music channel or genre can be a music channel or genre that is not pre-defined. In these implementations, the music channels can be newly generated by the system 100.

The music therapy application can be a web application, a mobile application, or a desktop application integrated with the streaming music service 104. Although FIG. 1 shows a separate application service 103, in some implementations, all functionality for the application are loaded on to the user device 101. Information about the user and the user's music therapy session can be stored in a database 105 that is either remote from the application or on the same device 101.

In some embodiments, each music therapy device 101 can be remotely monitored by a remote device 120. The remote device 120 can also be a smartphone, a portable computer, or another type of electronic device that executes applications. The remote device 120 can connect to music therapy devices 101 using the music therapy application service 103. Additionally or alternatively, the remote device 120 may be able to communicate with the user device 103 using near field communication, Bluetooth, wi-fi, or another communication protocol.

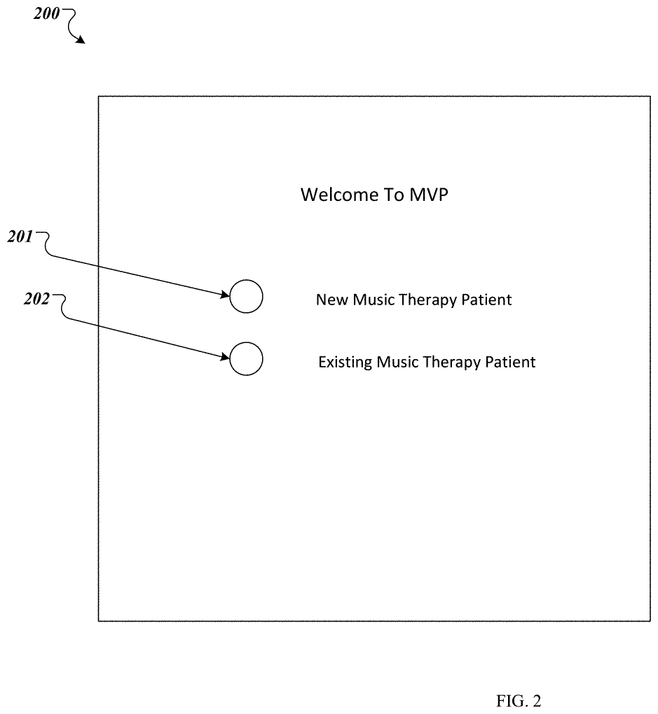

As disclosed above, a caregiver often initiates a music therapy session on behalf of a patient prior to providing the device 101 to the user 102. The music therapy application has an easy-to-use user interface for initiating music therapy sessions, managing, and gathering data about users before, during, and after music therapy sessions. The user interface will be described in more detail below with respect to FIGS. 2-7.

The music therapy and healthcare correlation system 100 of FIG. 1 also includes a medical information application service 106 that provides information about patients' medical care and treatment as well as information about patients' overall health and wellness. This information can be correlated with information from the music therapy application service 103 using a correlation service 108 in order to determine the effectiveness, or efficacy, of music therapy on a patient's health. The correlation process will be described in more detail below.

FIG. 2 illustrates an exemplary startup user interface for a music therapy device, e.g., user device 101, to initiate a music therapy session 200. This user interface is part of a music therapy application provided by a music therapy application service, e.g., the music therapy application service 103 of FIG. 1. At this initial user interlace, a user, who is most often the caregiver of the music therapy patient, chooses whether the patent is a new music therapy patient 201 or an existing music therapy patient 202.

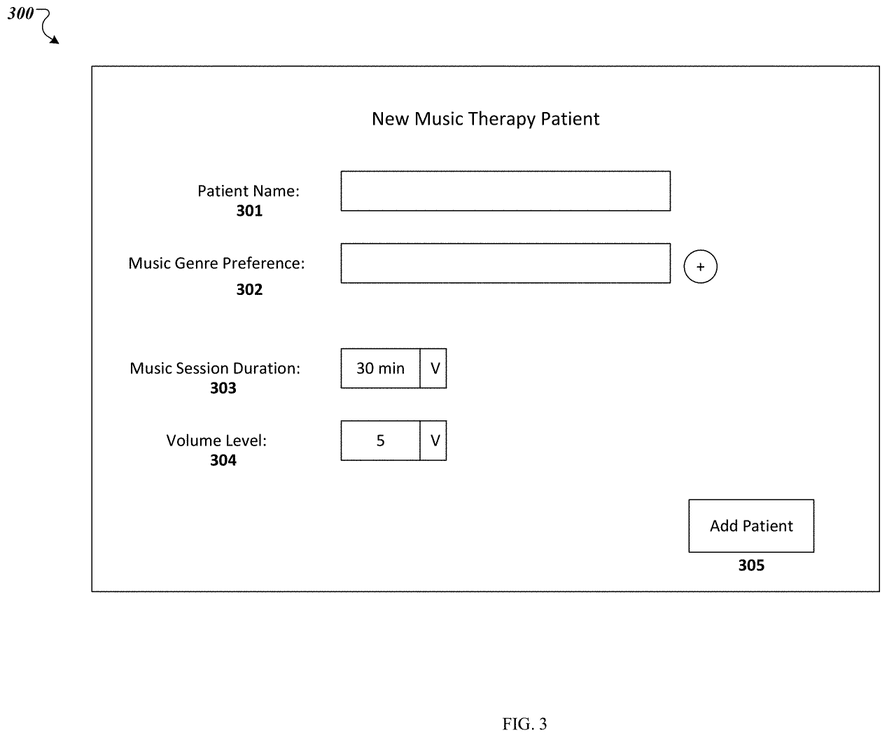

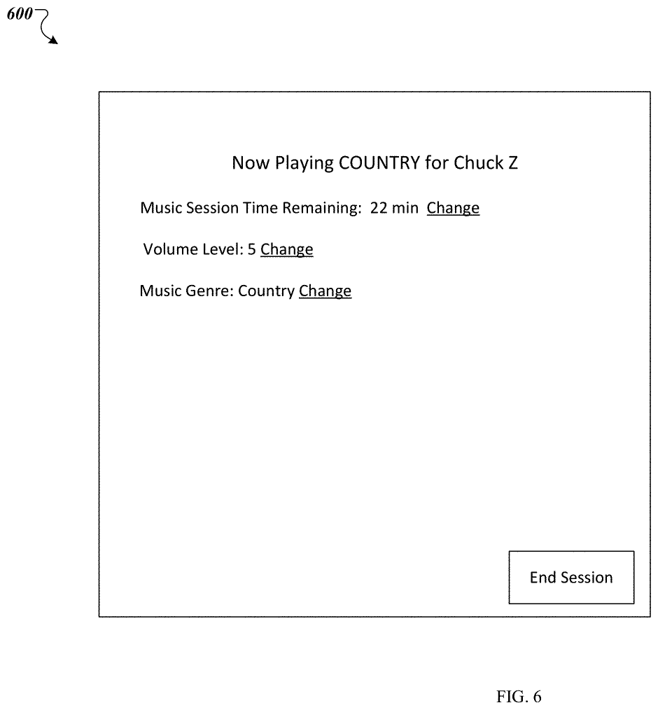

FIG. 3 illustrates an exemplary user interface for adding a new music therapy patient profile to the music therapy application 300. Patient profiles may be setup on device or off-device, remotely. This user interface is part of the music therapy application provided by the music therapy application service, e.g., the music therapy application service 103 of FIG. 1. When creating a profile for a new patient, the music therapy application 103 allows a user, i.e., the caregiver, to enter the new patient's name 301. The caregiver will also choose the music genre preferences of the new patient 301 by inputting the relevant information via user interface element 302, which may be, for example, a drop-down menu presenting various music genre options, e.g., electronic dance music, heavy metal, musical theater, jazz, rock, classical music, blues, popular music, pop music, hip hop music, folk music, country music, and reggae, to the user. The caregiver can add one or more genres for the new patient by interacting with a portion of the user interface element 302, such as, for example, a graphical button displaying an “+”. For each genre chosen, the music therapy application creates or obtains a streaming radio station from the streaming audio service 104 that matches the chosen genre. In some implementations, the music therapy application uses an application programming interface (API) from the streaming audio service 104 to request specific genres of music from the streaming audio service 104. In some implementations, the music therapy application can interact with media, e.g., music, in various ways. For example, where the music therapy application streams music from the streaming audio service 103, the music therapy application can exchange coded polls/responses with a server system of the streaming audio service 103 to obtain data for streaming music. As another example, where the music therapy application uses media data that are stored in a user device or an external memory of the user device, the data for the media can be triggered to be uploaded for presentation to a user. The music therapy application will use these chosen genres to provide music to a user during music therapy sessions as described in more details with regard to FIG. 6. The caregiver will also choose a default length of time for each music therapy session 303 for the new patient. For example, as illustrated in FIG. 3, the default duration for a music therapy session is 30 minutes. The default setting can change by the caregiver interacting with the graphical user interface element 303, which may be a pre-populated drop-down menu, a textual input box, or some other interface element that allows the user to specify the amount of time a music session should last.

The caregiver will set a default volume level 304 at which the music will play for the patient. The caregiver can change the default setting by interacting with the graphical user interface element 304, which may be a pre-populated drop-down menu, a textual input box, or some other interface element that allows the user to specify, the volume level at which music should play for the user. Once the caregiver has entered the new patient information, the caregiver can add the new patient with the “Add Patient” button 305. The system stores the new patient information in a database, e.g., database 105 of FIG. 1.

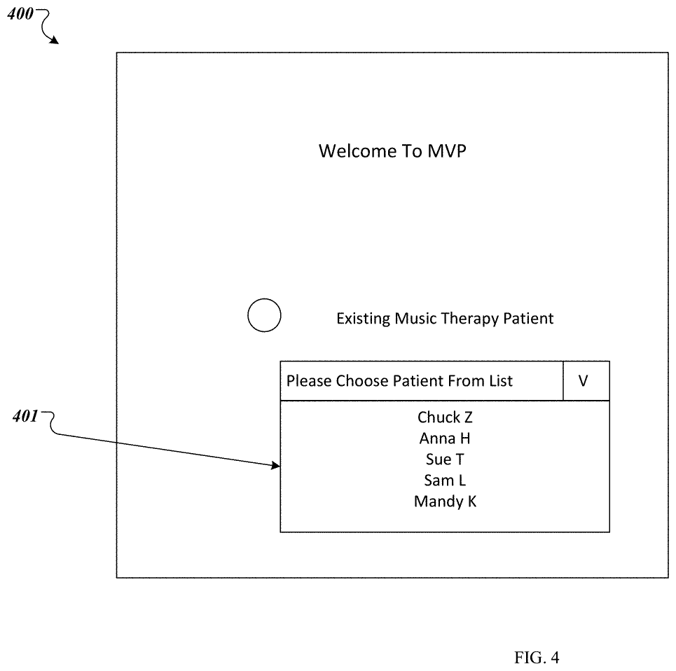

When a patient's profile already exists in the music therapy application, the caregiver can choose “existing user” when initiating a music therapy session. FIG. 4 illustrates a user interface for choosing an existing music therapy patient 400. The music therapy application provides a way for the caregiver to access a profile of an existing user through the user interface. For example, as illustrated in FIG. 4, an option list of existing patients may be displayed to the user 401. The music therapy application provides the list of existing patients from the users stored in the database, e.g., database 105 of FIG. 1. The system may additionally or alternatively present the caregiver with a textbox where the caregiver can start typing the name of the person for whom they would like to start a music therapy session. The textbox may include a suggestion of users whose names match the characters typed into the textbox. Once the caregiver has found the right existing user, the caregiver can choose the desired patient from the list or suggestions. For example, as illustrated in FIG. 4, the user may choose “Chuck Z.”

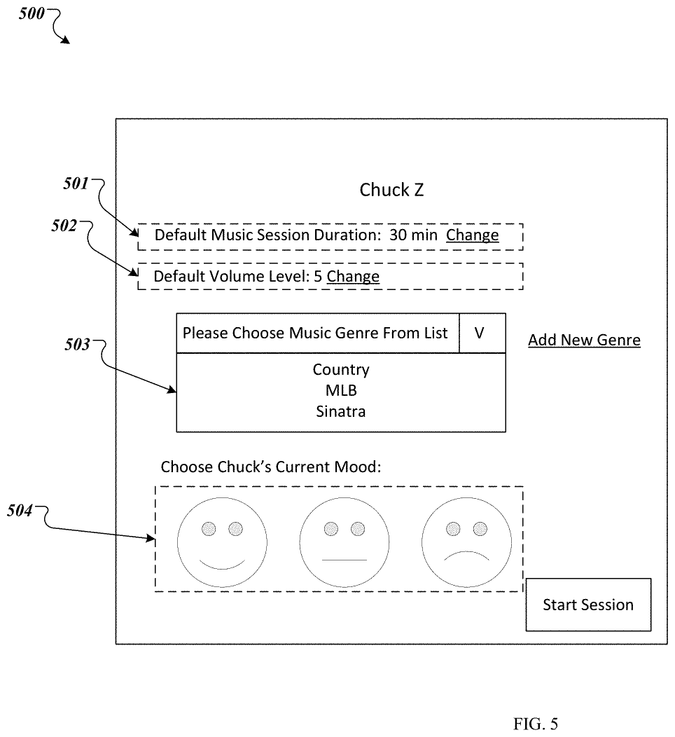

FIG. 5 illustrates an exemplary music therapy session initialization for a specific user 500. The system uses profile information of a chosen user stored in the database 105 to populate the form. When the requested user is chosen, the user device 101 sends the requested user name or an identifier identifying the requested user to the music therapy application service 103. The music therapy application service 103 looks up the requested user in the database 105 and obtains additional information about the user's preferences and music therapy sessions. In this case, the music therapy session form is pre-populated with Chuck Z's name, the default duration of his music therapy sessions 501, and his default preferred volume level 502. All of this information is stored in the database 105 when the user is first added to the system and can be updated by editing the preferences of the user in the music therapy application. For example, by clicking on the “change” link next to user interface element 502 for default volume level, a caregiver may be able to change the default volume level ter music therapy sessions associated with user “Chuck Z.” The music therapy application may have a separate user interface for editing user preferences and information. After editing, the user information may be sent from the user interface executing the client device to the music application server. The information may then be stored in the database as default information for the user.

In addition to setting the music session duration and volume level, the caregiver can then choose the music genre to play for the patient using the predefined stations that were previously set for the patient 503. The music therapy application will obtain streaming music from the streaming music audio service 104 that matches the music genre chosen to play for the given music therapy session.

The caregiver can also record the patient's, e.g., Chuck's, mood prior to starting the music therapy session. As shown in FIG. 5, in some implementations, the caregiver can record the patient's mood using graphical representations of emotional states 504, e.g., emojis. FIG. 5 illustrates three emotional states: “happy,” “neutral,” and “sad.” In other implementations, the caregiver can record the patient's mood with words or other mood-determining methods. By using emojis, the system 100 can increase user engagements.

Once the patient's mood is chosen, the caregiver can start the session using a “Start Session” button and hand the device over to the patient. In some implementations, the device has a lanyard attachment with an easy breakaway so that the patient can wear the device around his or her neck. FIG. 1 shows user device 101 with a lanyard 115. This lanyard may be made out of nylon, cord, twill, fabric, or another material that is lightweight, yet can hold the weight of a user device, e.g., device 101, around a person's neck. The lanyard may have a quick release metal or plastic clip so that the lanyard can quickly be released from the user's neck and the device can be retrieved.

The device may also be locked down so that the only application the patient can access on the music therapy device is the music therapy application. A caregiver or other supervisor users can enable restrictions on the user device in order to disable other device functionality, e.g., calling, texting, browsing the Internet, or playing games. The functionality may be disabled for only the duration of the music therapy sessions or the functionality may be disabled for all devices running the music therapy application. Device administrators, caregivers, or other supervisor users can use a range of techniques to configure policies and apply device restrictions. For example, a device administrator for smartphones may use a management framework for the device type to configure and update settings, deploy applications, monitor compliance, query devices, and remotely wipe or lock devices. Additionally or alternatively, devices may have an administrative password that is required to do anything on the device except listen to the music therapy session. All other functionality may require the inputting of the password.

In some implementations, once the caregiver provides the music therapy device to the patient for the music therapy session, the caregiver can remotely monitor the status of the music therapy session, e.g., using remote device 120 of FIG. 1. As disclosed above, the remote device may connect to the music therapy application service 103 in order to obtain information about the devices currently running music therapy applications or the remote device may connect directly with a user device 101 executing the music therapy, application using a communication protocol. The caregiver may be able to view the status of a music therapy session, the current genre of music to which the patient is listening and the volume at which the patient is listening to the music. The caregiver can also remotely start and stop a particular music therapy session, adjust the volume of the music, or change the station which the patient is presently listening. Remote instructions can be sent from the remote device to the user device directly or through the music therapy application service. In order to monitor the patient's music therapy session, the caregiver, or other user, can execute an instance of the music therapy application on a second device and connect to the patient's device by wireless or wired communication using, e.g., BlueTooth, near field communication, a network, or the Internet.

In some implementations, the caregiver may also monitor health information and geographic location of the patient. For example, the caregiver may record the blood pressure of a user prior to a music therapy session and note that the patient is in the cafeteria of her care home in Los Gatos, Calif. Geographic locations can include addresses as well as specific places within a building.

In operation, the patient may be handed the music therapy device 101 by the caregiver after the caregiver has initiated the music therapy session. After initiation, the music therapy application starts playing the chosen music at aloes volume, e.g., at a level 0. In some implementations, the music therapy application starts playing the chosen music upon receiving user input by a user or a caregiver. For example, a user or a caregiver can press a particular icon provided by the music therapy device 101 to start playing the chosen music. As another example, a user or a caregiver can start playing the chosen music remotely. In particular, a user or a caregiver can operate a central management interface provided by a remote device to start playing the chosen music. The music therapy application service 103 may acquire the patient's preferences from the database 105. The music therapy application service 103 sends information to the user device 101 to gradually and incrementally increase the volume of the music playing on the user device 101 until the music volume level of the device 101 is at the predetermined volume chosen by the caregiver, a staff, or a patient during the music therapy session initiation or adjusted after initial setup to a patient's preferred final music volume level. To increase the volume, any suitable techniques can be used. For example, the volume controls of the music therapy application may increase the volume in slowly increasing increments, approximately 1 level per 2-3 seconds. As another example, the volume can be incrementally increased at pre-defined intervals. By increasing the volume to a comfortable level in these ways, the system 100 can provide well-being to a user of the user device 101.

FIG. 6 illustrates an exemplary user interface of the music therapy device 101 that the music therapy application displays during a music therapy session. The music therapy application plays music from the streaming audio service 104 that matches a chosen music genre for the music therapy session. As illustrated, the user interface 600 might show the time remaining for the music therapy session, the volume level of the music, the title and artist of the current song, and the currently playing music genre. The music therapy application executing on the user device 101 can count down the time left of the music therapy session. The volume level displayed is the current listening volume level. This level may change any time a caregiver or other user adjusts the volume either on the device 101 or by a remote device 120. Information about the currently playing song including music genre, title, and artist which the music therapy application is playing from the user device. The interface also provides the patient or a caregiver with the ability to change any facet of the app's settings, including the remaining time of the music session, the volume level of the music, or the music genre on the device itself or remotely as described above.

The patient may be able to change information about the music therapy session through the user interlace 600 of the music therapy application. Some changes may only require client side code of the music therapy application executing on the user device, Other changes may go through the music therapy application service to take effect. A caregiver may either directly or remotely make changes to the patient's music therapy session while the session is in progress. Remote interaction with the user device is disclosed above with respect to FIG. 1. The patient or caregiver also has the option of ending the session prior to the music session's time running out.

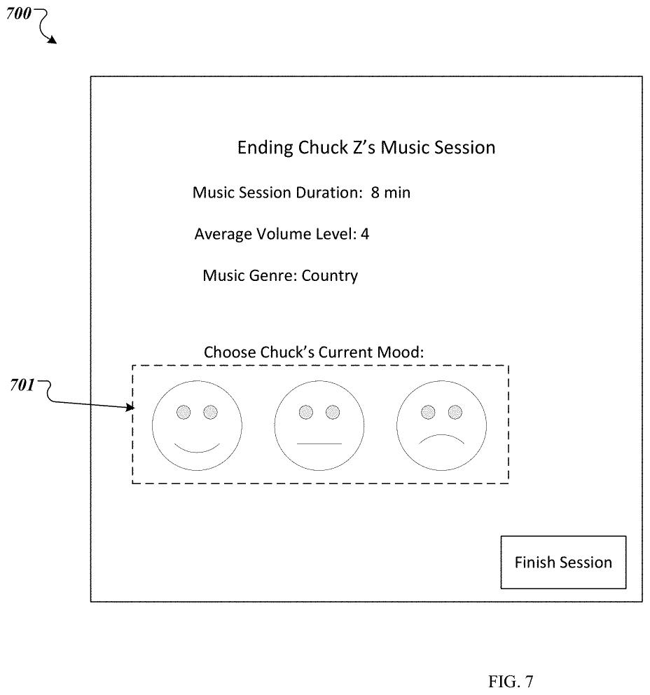

FIG. 7 illustrates an exemplary user interface of the music therapy device 101 at the end of a music therapy session 700. At the end of a music therapy application, the music therapy application displays an ending user interface from the music therapy application service. As illustrated, the system displays the music session duration, the average listening volume, and the music genre or genres to which the patient listened during the music therapy session. The system may also display a list of songs, including title and artist, that were played during the music therapy session. This data may have been stored in the database 105 during the music therapy session and associated with a music therapy session identification number. The music play list may also include the duration of each song and the duration that each song was played during the music session. The music therapy application running on the user device 101 gathers the data during the music therapy session and transmits it to the music therapy application service 103 which then stores the session data in the database 105. The caregiver also records information about the patient, including the patient's mood, after the music therapy session 701. The mood information is associated with the music therapy session identification number and also stored in the database 105.

The music therapy application running on the user device 101 can capture additional personal information about patients who listen to music during music therapy sessions including blood pressure and heart rate when listening to music. In some implementations, this additional personal information can be input into a user interface. The music therapy application service may send instructions to the music therapy application running on the user device 101 to collect the information. In some embodiments, the music therapy application executing on the user device 101 may let a caregiver enter information that is collected using a blood pressure machine or heart rate monitor. In other embodiments.

In addition to personal metrics, the music therapy application will capture listening metrics through client-side code executing on the user device 101, send the metrics to the music therapy application service 103, and store listening metrics in the database 105. For example, the listening metrics may include the date and time of day of each music therapy session, the duration of the music therapy session, the genre of music playing during the music therapy session, the average volume of the music during the music therapy session, and the location of the music therapy session. In some implementations, the music therapy application can capture who initiates or ends a music therapy session, and send the information to the music therapy application service 103. The music therapy application can obtain this information, who initiates or ends a music therapy session, by tracking log-in and log-off records. For example, when a particular caregiver logs in the music therapy application using his/her particular identification code, the music therapy application can determine that the particular caregiver has initiated a music therapy session. When another caregiver logs off from the music therapy application using his/her particular identification code, the music therapy application can determine that the caregiver has ended a music therapy session.

The music therapy application service 103 can combine information from the patient's music therapy sessions received from client-side code of the music therapy application executing on the user device 101 with medical data of the patient from a medical information application service 106 as shown in FIG. 1. The medical information application service 106 can contain information about the patient such as the patient's current medicines and dosages, the time of day that the medicines are administered, the patient's daily activities, the patient's vital signs, the patient's food in-take, and any other health-related information that is tracked by medical personnel.

The music therapy and healthcare correlation system 100 uses medical data from the medical information application service 106 along with music therapy data from the music therapy sessions to determine trends and correlations between music therapy sessions and patient medical changes and improvements using a correlation service 108. The patient may experience changes in medicine dosages, daily activity habits, food intake, sleeping patterns, and moods. For example, the data may indicate that after each day of Chuck having music therapy, Chuck needs 5 mg less of his dosage of psychotropic drugs. Chuck may also only require the lower dose of the drugs as long as he has had a music therapy session within 30 hours of the dosage distribution. Therefore, caregivers should know to provide Chuck with music therapy on a regular basis, within a 30-hr time span, so that he can maintain his lower drug dosage requirement.

In some implementations, the system records information about a patient's music therapy sessions for a determined period of time while the patient is running a music therapy application on his or her user device 101. The music therapy application can store the recorded information in a database, e.g., database 105. The application service can then access the data from the database 105 so that the personal information and listening information from multiple sessions can be correlated, using a correlation service 108, with the patient's medical information, from the medical information application service 106, for that determined period of time. The music therapy and healthcare correlation system can more accurately determine the efficacy of music therapy for a patient with more data received and processed from music therapy sessions and healthcare data collected by healthcare professionals.

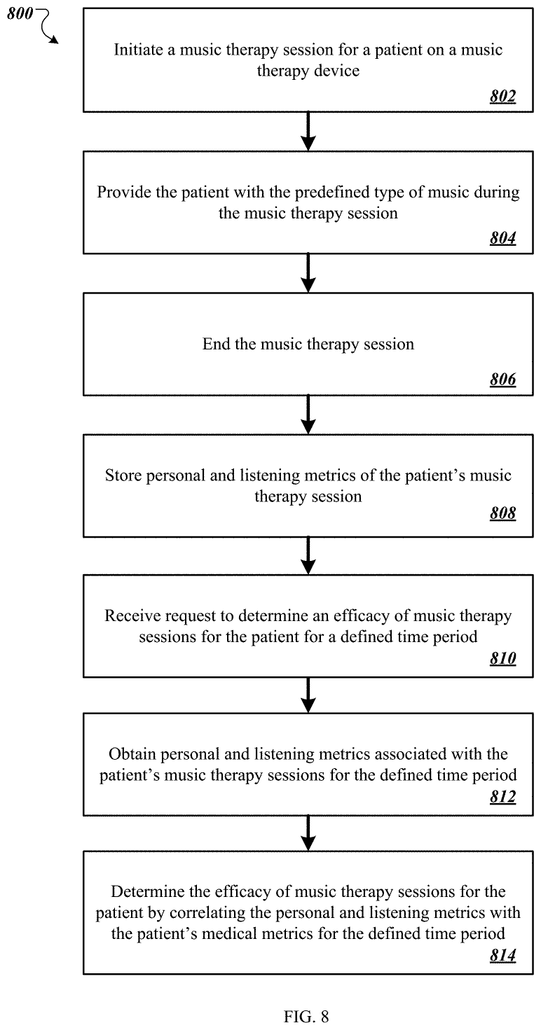

FIG. 8 is a flowchart of an example process 800 for determining an efficacy of music therapy sessions for a patient. For convenience, the process 800 will be described as being performed by a system of one or more computers, located in one or more locations, and programmed appropriately in accordance with this specification. For example, a music therapy and healthcare correlation system, e.g., the music therapy and healthcare correlation system 100 of FIG. 1, appropriately programmed, can perform the process 800.

The music therapy application, using a music therapy application service 103, can initiate a music therapy session for a patient on a music therapy device 802. The music therapy device is configured to execute a music therapy session that includes playing a predefined type of music for a predefined amount of time at a specified volume 804. After initialization, the system provides the patient with the predefined type of music during the music therapy session. Although the music therapy session has a predefined time period, a user may stop the music therapy session any time prior to the end of the predefined time period. After either the predefined amount of time has passed or a user requests an end to the music therapy session, the system ends the Music therapy session 806. The system then stores personal and listening metrics of the patient's music therapy session. As illustrated in FIG. 1, these metrics may be stored in a database.

Upon receiving a request to determine an efficacy of music therapy sessions for the patient for a defined time period 810, the system obtains personal and listening metrics associated with the patient's music therapy sessions for the defined time period 812. For example, a healthcare provider may want to determine the efficacy of the music therapy sessions for a patient within the last month. The system then provide personal and listening metrics for each music therapy session that the patient has had in the last month. The system determines the efficacy of music therapy sessions for the patient by correlating the personal and listening metrics of the patient's music therapy sessions with the patient's medical metrics for the defined time period. The system can then display the correlated data to a user, e.g., the healthcare provider, for further analysis. The healthcare provider can view the changes in mood, activity level, eating habits, sleeping patterns, and overall wellness attributable to the music therapy sessions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them, Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the s device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular so order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method for determining an efficacy of music therapy, the method comprising:
initiating a music therapy session for a patient on a music therapy device, the music therapy device configured to execute a music therapy session that includes playing a predefined type of music for a predefined amount of time at a specified volume;
providing the patient with the predefined type of music during the music therapy session;
ending the music therapy session;
storing personal and listening metrics for the music therapy session;
receiving a request to determine an efficacy of music therapy sessions for the patient for a defined time period;
obtaining personal and listening metrics associated with the patient's music therapy sessions for the defined time period; and
determining the efficacy of music therapy sessions for the patient by correlating the personal and listening metrics with the patient's medical metrics for the defined time period.

2. The method of claim 1, wherein initiating a music therapy session for a patient on a music therapy device includes receiving an indication of an initial mood of the patient.

3. The method of claim 2, wherein ending a music therapy session includes identifying a post music therapy mood of the patient.

4. The method of claim 3, further comprising:
determining a change in a mood of a patient attributable to the music therapy session based on a difference between the initial mood of the patient and the post-therapy mood of the patient.

5. The method of claim 3, wherein determining the efficacy of music therapy for the patient comprises correlating the patient's change in mood with medical metrics.

6. The method of claim 1, wherein medical metrics comprise medications and dosages that the patient is currently taking, sleep patterns, eating habits, or daily activity habits.

7. The method of claim 1, wherein personal metrics comprise, for each music therapy session in the defined period of time: heart rate data, mood data, or blood pressure data.

8. The method of claim 1, wherein listening metrics comprise for each music therapy session in the defined period of time: the date and time of day of the music therapy session, the duration of the music therapy session, the genre of music to which the patient listened, the location of the music therapy session, who engaged with the patient to initiate and/or end the music therapy session, and the average volume at which the patient listened to the music.

9. The method of claim 1, wherein providing the patient with the predefined type of music during the music therapy session comprises:
accessing a preferred final music volume level of the patient;
initializing the music therapy device with music at a low volume automatically; and
incrementally increasing the volume on pre-defined intervals until the music volume matches the preferred final music volume level of the patient.

10. The method of claim 1, further comprising:
providing information regarding the efficacy of the music therapy sessions for display to a user.

11. A system comprising:
one or more computing devices; and
at least one non-transitory computer-readable device storing instructions that are executable by the one or more computing devices to perform operations comprising:
initiating a music therapy session for a patient on a music therapy device, the music therapy device configured to execute a music therapy session that includes playing a predefined type of music for a predefined amount of time at a specified volume;
providing the patient with the predefined type of music during the music therapy session;
ending the music therapy session;
storing personal and listening metrics for the music therapy session;
receiving a request to determine an efficacy of music therapy sessions for the patient for a defined time period;
obtaining personal and listening metrics associated with the patient's music therapy sessions for the defined time period; and
determining the efficacy of music therapy sessions for the patient by correlating the personal and listening metrics with the patient's medical metrics for the defined time period.

12. The system of claim 11, wherein initiating a music therapy session for a patient on a music therapy device includes receiving an indication of an initial mood of the patient.

13. The system of claim 12, wherein ending a music therapy session includes identifying a post-music therapy mood of the patient.

14. The system of claim 13, wherein the operations further comprise:
determining a change in a mood of a patient attributable to the music therapy session based on a difference between the initial mood of the patient and the post-therapy mood of the patient.

15. The system of claim 13, wherein determining the efficacy of music therapy for the patient comprises correlating the patient's change in mood with medical metrics.

16. At least one non-transitory computer-readable storage device storing instructions that, when received by one or more computing devices cause the one or more computing devices to execute operations comprising:
initiating a music therapy session for a patient on a music therapy device, the music therapy device configured to execute a music therapy session that includes playing a predefined type of music for a predefined amount of time at a specified volume;
providing the patient with the predefined type of music during the music therapy session;
ending the music therapy session;
storing personal and listening metrics for the music therapy session;
receiving a request to determine an efficacy of music therapy sessions for the patient for a defined time period;
obtaining personal and listening metrics associated with the patient's music therapy sessions for the defined time period; and
determining the efficacy of music therapy sessions for the patient by correlating the personal and listening metrics with the patient's medical metrics for the defined time period.

17. The device of claim 16, wherein initiating a music therapy session for a patient on a music therapy device includes receiving an indication of an initial mood of the patient.

18. The device of claim 17, wherein ending a music therapy session includes identifying a post-music therapy mood of the patient.

19. The device of claim 18, wherein the operations further comprise:
determining a change in a mood of a patient attributable to the music therapy session based on a difference between the initial mood of the patient and the post-therapy mood of the patient.

20. The device of claim 18, wherein determining the efficacy of music therapy for the patient comprises correlating the patient's change in mood with medical metrics.
